(19) United States
(12) Patent Application Publication
(10) Pub. No.: US 2019/0046149 A1
(43) Pub. Date: Feb. 14, 2019

(54) METHOD FOR CREATING A VOLUME IMAGE AND X-RAY IMAGING SYSTEM

(71) Applicant: Siemens Healthcare GmbH, Erlangen (DE)

(72) Inventors: Oliver Hornung, Dormitz (DE); Markus Kowarschik, Nürnberg (DE)

(21) Appl. No.: 16/100,416

(22) Filed: Aug. 10, 2018

(30) Foreign Application Priority Data
Aug. 11, 2017 (EP) 17185956.4

(51) Int. Cl. A61B 6/00; G06T 11/00; G06T 7/20

(52) U.S. Cl. CPC A61B 6/5264; G06T 7/20; G06T 11/005

(57) ABSTRACT

Methods and Systems are provided for creating a volume image of a moving object under examination in a selected state of motion from a series of projection images captured during rotational motion of an image-capture system about the object under examination in each case with a plurality of projection images from different projection directions. The series of projection images are acquired. A first state of motion is selected by selecting a first projection image that images the first state of motion. A consistency or redundancy condition is determined. Further projection images are identified that image the first state of motion, by applying the consistency or redundancy condition to the projection images of the series. The identified projection images and the first projection image are reconstructed to yield a reconstructed volume image.

### CROSS REFERENCE TO RELATED APPLICATIONS

This application claims the benefit of EP17185956.4 filed on Aug. 11, 2017, which is hereby incorporated by reference in its entirety.

### FIELD

Embodiments relate to a method for creating a volume image of an object under examination in a desired state of motion an X-ray imaging system.

### BACKGROUND

Time-resolved 3D X-ray imaging (4D imaging) with high soft tissue contrast using a C-arm X-ray imaging system is currently possible, for example assuming both periodic and sufficiently smooth fields of motion. A series of projection data sets is captured for a generally periodically moving object under examination while the C-arm rotates about the object under examination, e.g. from a plurality of projection directions of the C-arm relative to the object under examination. The series includes at least half a rotation run about the object under examination.

When reconstructing the projection data sets, a problem arises that the object under examination is in different states of motion in successive projection images. In order to be able to reconstruct the corresponding matching states of motion into volume images, the states of motion must be detected and assigned to the respective projection images. This is highly complex and is very imprecise in the case of rapid movements. The detection of states of motion is performed for example using optical tracking in a case of respiratory movements or EMT in the case of heartbeats. A further problem includes that with known C-arms continuous rotation is impossible, a sequence of forward-backward rotations taking place instead, leading to a restriction of the angular range and restricted dynamics resulting from mechanical instabilities of the C-arm. The latter problem may be solved for example by introducing a slip ring on the C-arm suspension, so as to make continuous rotation possible, see for example also the patent application “Method for capturing an image data set with an X-ray imaging system and X-ray imaging system” filed at the same time.

### SUMMARY AND DESCRIPTION

The scope of the present disclosure is defined solely by the appended claims and is not affected to any degree by the statements within this summary The present embodiments may obviate one or more of the drawbacks or limitations in the related art.

Embodiments provide a method that allows a simple time-resolved 3D reconstruction from a series of projection images without including additional measurements of states of motion.

Embodiments relate to the tomographic reconstruction of time-resolved volumetric images with high soft tissue contrast, either entirely without the use of contrast agent (for example representation of the bronchial tubes during endobronchial or percutaneous lung interventions) or in the case of (quasi-)steady-state contrast agent perfusion (for example representation of the liver during oncological interventions), e.g. without significant contrast agent dynamics.

A method is provided for creating a volume image of a moving object under examination in a selected state of motion from a series of projection images captured during rotational motion, for example including of at least half a rotation run, of an image-capture system about the object under examination, in each case with a plurality of projection images from different projection directions. The method includes: providing the series of projection images, selecting a first state of motion for example, by selecting a first projection image that images the first state of motion, providing or determining a consistency or redundancy condition, identifying further projection images, that image the first state of motion, by applying the consistency or redundancy condition to the projection images of the series, and reconstructing a volume image from the identified projection images and the first projection image.

The series of projection images is or was captured, for example, during at least half) (180°) or more (for example 270°) of a rotation run or a whole run or more, for example two, rotation runs of an image-capture system about the object under examination. A<180° run is also possible; at least 180° plus the opening angle of the X-ray, however, may be necessary. Movement of the object under examination may be periodic. Periodic or partially periodic motion such as for example respiratory cycles or heartbeats improves the quality of the reconstruction result. In the case of periodic motion, where possible at least two, for example, a plurality of motion cycles may be captured, so that at least two matching states of motion are present.

Selection of a state of motion may be performed manually for example by manual user input, e.g. a user selects from the series a projection image of the desired state of motion. A given state of motion (for example maximum inhalation during respiratory motion, first image of the series etc.) may also be automatically predetermined. Determination of a consistency or redundancy condition may for example be calculated as described in the article Rigid Motion Compensation in Interventional C-arm CT Using Consistency Measure on Projection Data, R. Frysch and G. Rose, MICCAI 2015, Part I, LNCS 9349, pp. 298-306, 2015. Various possible ways of calculating such consistency or redundancy conditions are known.

A consistency or redundancy condition may also be provided that was previously stored, for example. Using the consistency or redundancy condition, all the projection images of the series of projection images that were captured in the same state of motion are then determined by filtering. There are at least two projection images, for example, a plurality of projection images from different projection directions, as it may be very improbable that the projection directions match. A volume image in a specific state of motion is reconstructed from the originally selected projection image and identified by the consistency or redundancy condition.

The method is purely image-based. No further measures are taken in addition to actual image acquisition to be able to calculate a time-resolved series of volume data sets. No separate hardware is needed for detecting the state of motion of the object under examination (patient), such as for example a belt for detecting respiratory motion when scanning the liver. The method may be carried out quickly, simply and easily and also subsequently if the series of projection images is already available.

According to one embodiment, reconstruction is carried out by an iterative reconstruction method, for example, an ART (Algebraic Reconstruction Technique) or SART (Simultaneous Algebraic Reconstruction Technique) technique. Such methods are known from the literature. Iterative reconstruction methods do not require equidistant distribution of the projection images along the trajectory of the rotation run of the image-capture system, as is ideally the case for example with conventional analytical reconstruction methods (e.g. the FDK method), that is based on numerical integration.

According to an embodiment, the consistency or redundancy condition includes a predeterminable tolerance threshold value with regard to the state of motion. Projection images with states of motion deviating at most by the tolerance threshold value from the state of motion are also identified and used for the reconstruction. More projection images are available, that brings about a qualitatively better reconstruction result, that may however be accompanied in part by motion blur.

According to an embodiment, a plurality of volume images of an object under examination are determined in multiple, e.g. successive, selected states of motion. For example, the object under examination may also be reconstructed over a whole series of successive states of motion, for example in the case of respiratory motion from the state of maximum exhalation to the state of maximum inhalation. The volume images may be combined into a time-dependent image data set, for example a video sequence.

A system is provided for carrying out the method. The system includes an X-ray imaging system including an image-capture system with an X-ray detector and an X-ray source, that are arranged on a mount that is configured to rotate about an object under examination capturing a plurality of projection images from different projection directions in the process. The system includes a system controller for driving the X-ray imaging system, a computing unit for determining the consistency or redundancy condition, and an image processing system for processing projection images and for reconstructing the projection images into volume images. The mount is formed by a C-arm and the C-arm is suspended by a slip ring construction, so as to allow continuous rotation of the C-arm. The X-ray imaging system includes a memory unit for storing image data and/or calculation data. The X-ray imaging system includes an input unit for accepting user input. Alternatively, a computed tomography system may also be used.

The X-ray imaging system may include when rotating, a smooth field of motion or a mechanism optimized with regard to deviations.

### BRIEF DESCRIPTION OF THE FIGURES

FIG. 1 depicts the sequence of a method according to an embodiment.

FIG. 2 depicts a diagram corresponding to the sequence of the method of FIG. 1 with resultant projection images and volume images according to an embodiment.

FIG. 3 depicts a view of a C-arm X-ray imaging system with a slip ring according to an embodiment.

### DETAILED DESCRIPTION

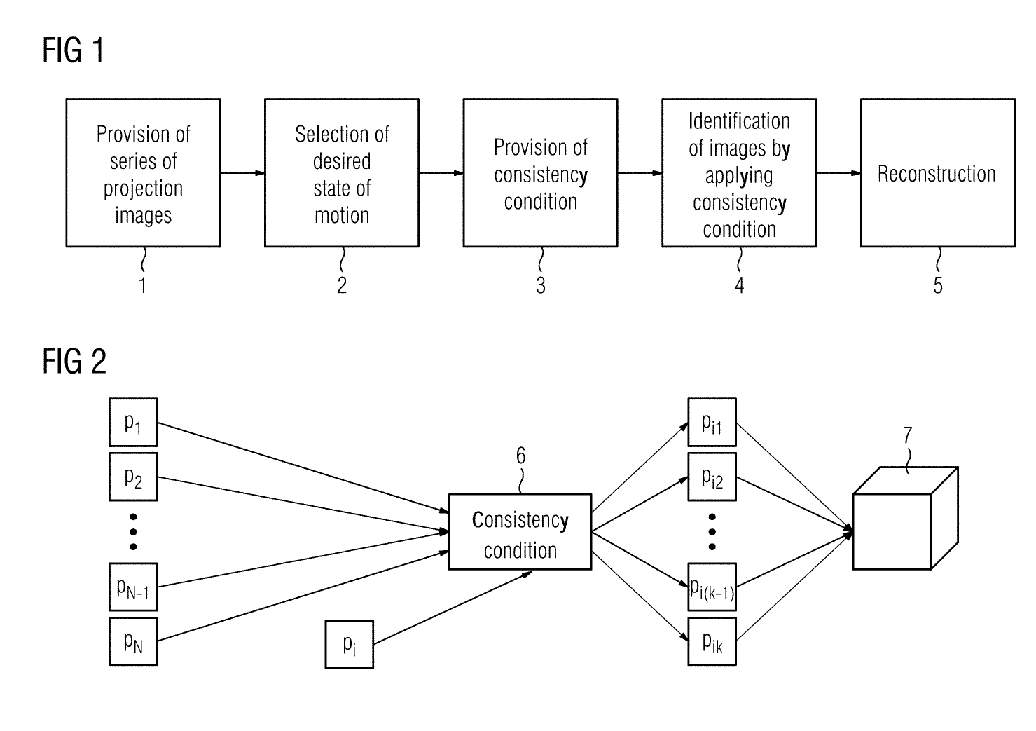

FIG. 1 depicts the order of a method with five acts, while FIG. 2 depicts the corresponding projection images or volume images determined from the respective acts.

At act 1 a series of projection images P₁, P₂, . . . P_{N-1}, P_{N} of an object under examination is captured or alternatively provided. The series may, for example, be captured during rotation of an image-capture system about the object under examination, but does not necessarily have to include a given angular sector or a specific number of rotation runs. The object under examination may pass, for example, through a cyclic motion (respiratory cycle, heartbeat) or a non-cyclic motion. Capture of the series P₁, P₂, . . . P_{N-1}, P_{N} is carried out by an X-ray imaging system to be described further below. The series may also be captured prior to the start of the method and then provided at act 1, for example retrieved from a memory.

At act 2, a selection is made with regard to the desired first state of motion of the object under examination. Act 2 proceeds by selection of a first projection image Pᵢ, that images the first state of motion of the object under examination, from the series P₁, P₂, . . . P_{N-1}, P_{N}. Thus, for example, an operator may perform an input and select the first projection image Pᵢ, for example directly by clicking on the corresponding projection image. Alternatively, the corresponding state of motion may also be selected automatically, for example maximum exhalation or inhalation in the case of respiratory motion. For input by an operator, an input unit (keypad, touchscreen etc.) may be used.

At act 3, the consistency or redundancy condition 6 is provided or determined. Mathematical derivation of such a consistency or redundancy condition may be performed in accordance with the method in the article Rigid Motion Compensation in Interventional C-arm CT Using Consistency Measure on Projection Data, R. Frysch and G. Rose, MICCAI 6, Part I, LNCS 9349, pp. 298-306, 2015. Further options for determining consistency or redundancy conditions are known. Consistency or redundancy conditions 6 are based on that, in projection images associated with different angulations (projection directions of the image-capture system with regard to the object under examination), redundancies are present in the captured data.

In the above-mentioned publication, such a consistency condition is used to compensate patient motion, for example, to adapt the projection geometry (defined by a list of projection matrices) after acquisition of the projection images to the patient motion that has occurred, such that the resultant reconstruction result exhibits the fewest possible motion artifacts. In contrast, embodiments use the consistency or redundancy condition to identify suitable projection images and then reconstruct therefrom the moving object under examination in the desired state of motion. Therefore, in an embodiment no significant contrast agent dynamics may be visible in the projection images since the dynamics may cause inconsistency and so suggest violation of the consistency condition. that also applies in the event of the object under examination not moving at all.

The consistency or redundancy condition may also already have been previously determined and now be retrieved from a memory unit.

At act 4, further projection images Pᵢ₁, Pᵢ₂, . . . Pᵢ₍ₖ₋₁₎, Pᵢₖ are determined by applying the consistency or redundancy condition to the captured projection images of the series, for example, by a filter method, the further projection images imaging the first state of motion. The further projection images Pᵢ₁, Pᵢ₂, . . . Pᵢ₍ₖ₋₁₎, Pᵢₖ are determined or calculated retrospectively from the data set, without having to include further data, solely by filtering using the consistency or redundancy condition. The determined projection images Pᵢ₁, Pᵢ₂, . . . Pᵢ₍ₖ₋₁₎, Pᵢₖ, at least two, in general multiple projection images, all image the previously selected state of motion. If only a very few projection images may be determined, a predeterminable tolerance threshold value of the consistency or redundancy condition may be extended with regard to the state of motion, such that a larger number of projection images may be found. Although extending may lead to motion blur in the subsequent volume images, a higher number of projection images to be reconstructed increases the general volume image quality by minimizing undersampling artifacts. The tolerance threshold value may be user-selectable, e.g. is selected to be higher, or lower, by an input.

At act 5, the object under examination is reconstructed in the selected state of motion from the projection images identified. The first projection image P, and the determined projection images Pᵢ₁, Pᵢ₂, . . . Pᵢ₍ₖ₋₁₎, Pᵢₖ, that all image the first state of motion, are reconstructed into a first volume image 7. The reconstruction is performed for example with an iterative reconstruction method known from the literature (Algebraic Reconstruction Technique (ART), Simultaneous Algebraic Reconstruction Technique (SART)), that does not require equidistant distribution of the projection images along the trajectory of the scan.

According to an embodiment, the method may also be carried out repeatedly in succession and different volume images may thereby be generated that image multiple states of motion of the object under examination. In a captured respiratory cycle, a plurality of volume images may for example be generated between maximum exhalation and maximum inhalation. The volume images may be joined together into a sequence of volume images, for example, into a type of 3D video sequence.

Embodiments differ from the 4D DSA method, in which the contrast agent dynamics (based on a mask run and a perfusion run of the C-arm) are approximately reconstructed in time-resolved manner in a vessel system assumed to be rigid, that corresponds to a time-resolved representation of high contrast objects. Embodiments also functions differently from interventional dynamic perfusion imaging. In the latter, the contrast agent dynamics are reconstructed tomographically in time-resolved manner in the parenchyma (primarily in the brain in the event of an acute ischemic stroke). Patient motion is in this case corrected at best by rigid 2D/2D registration.

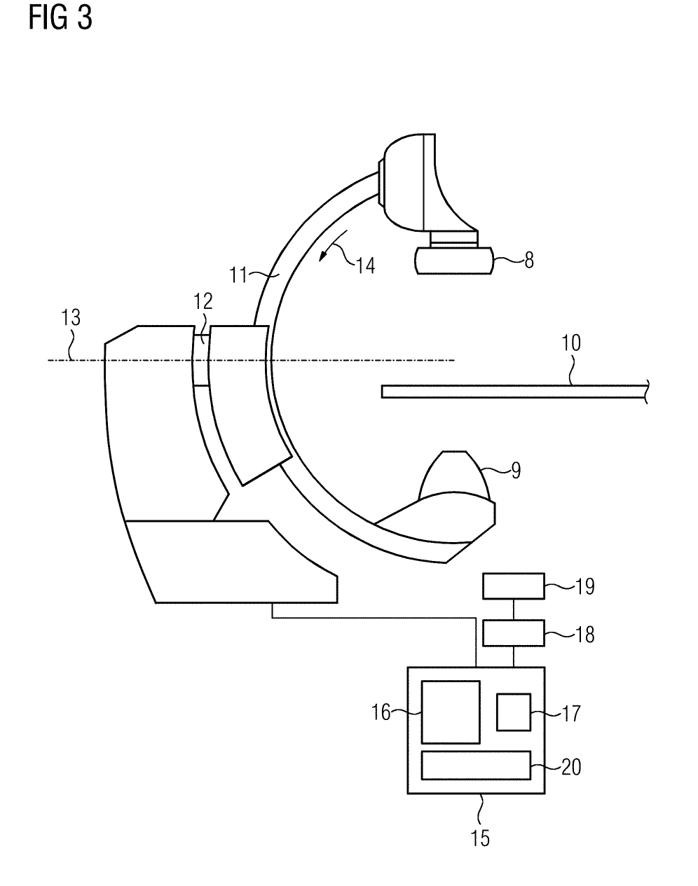

FIG. 3 depicts an X-ray imaging system with a C-arm 11, that is suitable for carrying out the method. An X-ray source 9 and an X-ray detector 8 are arranged on the C-arm 11. The C-arm 11 may be moved in multiple planes, inter alia the C-arm 11 may be moved translationally in the direction of the arrow 14 and rotated about the axis 13. During the rotation about an object under examination arranged on the patient table 10, a plurality of projection images of the object under examination may be captured from different projection directions (angulations). A slip ring is arranged by way of example in the suspension 12, by which slip ring the C-arm 11 may be rotated in continuous rotation about the axis 13. In previous X-ray imaging systems without a slip ring, the rotation was possible only up to at most one revolution, then it had to be rotated back. With the introduction of a slip ring in the rotational component of the angulation, the C-arm 11 may rotate without restriction. Different constant rotational speeds are straightforwardly possible. The field of motion of the X-ray imaging system is smooth due to use of the slip ring.

The X-ray imaging system includes a system controller 15 for driving purposes, and also an image processing system 15 with a computing unit 16 for determining the consistency or redundancy condition, for processing projection images and for reconstructing the projection images into volume images. The system controller 15 may for example be an image processing computer. The X-ray imaging system may additionally include a memory unit 17, an input unit 18, for example a keypad or a touchscreen, and a display unit 19, for example a monitor or a touchscreen.

Alternatively, the X-ray imaging system may also be a computed tomography system, where continuous rotation is also possible.

Other X-ray imaging systems are also possible for carrying out the method. The systems include constant rotational dynamics and thereby to exhibit the lowest possible level of mechanical instability when capturing the series of projection images, so that violation of the consistency or redundancy condition arises merely through the motion of the object under examination.

Embodiments provide for simplified time-resolved 3D X-ray imaging with high soft tissue contrast using a C-arm X-ray imaging system. A method is provided for creating a volume image of a moving object under examination in a selected state of motion from a series of projection images captured during rotational motion of an image-capture system about the object under examination in each case, in each case with a plurality of projection images from different projection directions, the method including: providing the series of projection images (P₁, P₂ . . . P_{N}), selecting a first state of motion for example, by selecting a first projection image that images the first state of motion, providing or determining a consistency or redundancy condition, identifying further projection images that image the first state of motion, by applying the consistency or redundancy condition to the projection images of the series, and reconstructing the identified projection images and the first projection image to yield a reconstructed volume image. The method functions algorithmically and purely on an image basis.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for creating a volume image of a moving object under examination in a selected state of motion from a series of projection images captured during rotational motion of an image-capture system about the object under examination, the method comprising:
acquiring the series of projection images comprising a plurality of projection images from different projection directions;
selecting a first state of motion by selecting a first projection image that images the first state of motion;
determining a consistency or redundancy condition;
identifying further projection images that image the first state of motion, by applying the consistency or redundancy condition to the projection images of the series; and
reconstructing the volume image from the identified projection images and the first projection image.

2. The method of claim 1, wherein the series of projection images was captured during at least half a rotation run of the image-capture system about the object under examination.

3. The method of claim 1, wherein reconstruction is carried out by an iterative reconstruction method.

4. The method of claim 3, wherein the iterative reconstruction method is an ART or SART technique.

5. The method of claim 1, wherein a plurality of volume images of an object under examination are determined in multiple selected states of motion.

6. The method of claim 5, wherein the plurality of volume images of an object under examination are determined in multiple selected successive states of motion.

7. The method of claim 6, wherein the plurality of volume images are combined into a time-dependent image data set.

8. The method of claim 1, wherein selection of the first state of motion is performed automatically or manually by user input.

9. The method of claim 1, wherein the consistency or redundancy condition includes a predeterminable tolerance threshold value with regard to the first state of motion.

10. An X-ray imaging system comprising:
an image-capture system configured to capture a plurality of projection images from different projection directions, the image-capture system comprising:
a mount configured to rotate about an object under examination;
an X-ray detector; and
an X-ray source;
wherein the X-ray detector and the X-ray source are arranged on the mount;
a system controller configured to control the image-capture system;
a computing unit configured to determine a consistency condition or a redundancy condition; and
an image processing system configured to process projection images and further configured to reconstruct the projection images into volume images.

11. The X-ray imaging system of claim 10, wherein the mount comprises:
a C-arm suspended by a slip ring construction, so as to allow continuous rotation of the C-arm.

12. The X-ray imaging system of claim 10, wherein the image-capture system is a computed tomography system.

13. The X-ray imaging system of claim 10, further comprising:
a memory unit configured to store image data, calculation data, or image data and calculation data.

14. The X-ray imaging system of claim 10, further comprising:
an input unit configured to accept user input.

15. The X-ray imaging system of claim 10, wherein the series of projection images was captured during at least half a rotation run of the image-capture system about the object under examination.

16. The X-ray imaging system of claim 10, wherein the image processing system is configured to reconstruct the projection images using an iterative reconstruction method.

17. The X-ray imaging system of claim 16, wherein the iterative reconstruction method is an ART or SART technique.

18. The X-ray imaging system of claim 10, wherein a plurality of volume images of an object under examination are captured in multiple selected states of motion.

19. The X-ray imaging system of claim 18, wherein the plurality of volume images of an object under examination are captured in multiple selected successive states of motion.
